(19) United States
(12) Patent Application Publication
(10) Pub. No.: US 2009/0012295 A1
(43) Pub. Date: Jan. 8, 2009

(54) Amorphous Erlotinib, processes for the preparation thereof, and processes to prepare additional forms of Erlotinib

(76) Inventors: Ales Gavenda, Ostrava - Lhotka (CZ); Augusto Canavesi, Lacate Varesino (CO) (IT); Dietmar Flubacher, Bad Krozingen (DE); Jiri Faustmann, Opava 6 (CZ); Alexandr Jegorov, Dobra Voda (CZ)

Correspondence Address: KENYON & KENYON LLP, ONE BROADWAY, NEW YORK, NY, 10004, US

(21) Appl. No.: 12/215,251

(22) Filed: Jun. 25, 2008

Related U.S. Application Data
(60) Provisional application No. 60/937,184, filed on Jun. 25, 2007.
(60) Provisional application No. 60/956,018, filed on Aug. 15, 2007.
(60) Provisional application No. 60/986,214, filed on Nov. 7, 2007.
(60) Provisional application No. 61/037,106, filed on Mar. 17, 2008.

(51) Int. Cl. C07D 239/72

(52) U.S. Cl. 544/293

(57) ABSTRACT

The present invention provides amorphous erlotinib, processes for the preparation thereof, and processes to prepare additional forms of erlotinib.

This application claims the benefit of U.S. Provisional Patent Application Nos. 60/937,184, filed Jun. 25, 2007; 60/956,018, filed Aug. 15, 2007; 60/986,214, filed Nov. 7, 2007; and 61/037,106, filed Mar. 17, 2008, the content of each of which is incorporated herein by reference in its entirety.

### FIELD OF THE INVENTION

The present invention is directed to amorphous erlotinib, processes for the preparation thereof, and processes to prepare additional forms of erlotinib.

### BACKGROUND OF THE INVENTION

Erlotinib, N-(3-ethynylphenyl)-6,7-bis(2-methoxyethoxy)-4-quinazolinamine, of the following formula

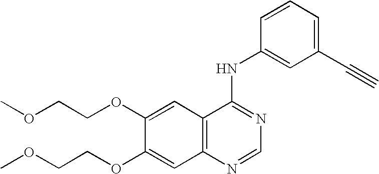

is the key intermediate of Erlotinib salts, such as Erlotinib HCl.

Erlotinib is administrated in a form of the HCl salt for treatment of patients with locally advanced or metastatic non-small cell lung cancer (NSCLC) after failure of at least one prior chemotherapy regimen. It is marketed under the trade name TARCEVA® by OSI Pharmaceuticals.

Erlotinib and its preparation are disclosed in U.S. Pat. No. 5,747,498; where the free base is produced, as shown in Scheme 1.

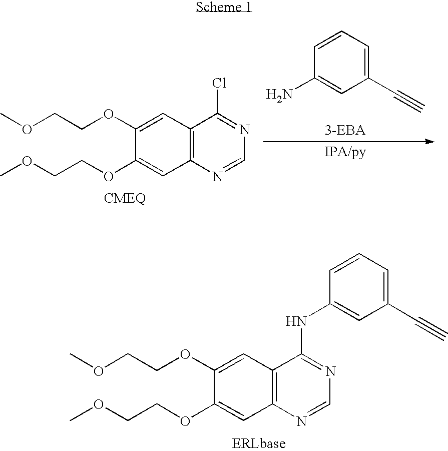

The reaction of 3-ethynylaniline (3-EBA) with 4-chloro-6,7-bis(2-methoxyethoxy)quinazoline (CMEQ) in a mixture of pyridine and isoproanol (IPA) yields erlotinib HCl (“ERL-HCl”), which transforms to the base by reacting it with aqueous solution of sodium bicarbonate in chloroform containing 10% of methanol. The free base is purified by chromatography on silica gel using a mixture of acetone and hexane.

U.S. Pat. No. 6,476,040 discloses methods for the production of Erlotinib and salts by treatment of 4-[3-[[6,7-bis(2-methoxyethoxy]-4-quinazolinyl]amino]phenyl]-2-methyl-3-butyn-2-ol with sodium hydroxide to obtain erlotinib base, as described in Scheme 2.

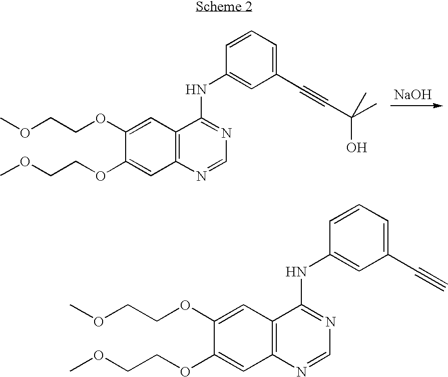

The preparation of Erlotinib is also disclosed in Molecules 2006, 11, 286. The process is done by extracting with dichlorormethane (DCM) a solution of Erlotinib hydrochloride after basification with concentrated ammonia, followed by evaporating the solvent to obtain a product having a melting point of 159 to 160° C.

Polymorphism, the occurrence of different crystal forms, is a property of some molecules and molecular complexes. A single molecule, like Erlotinib, may give rise to a variety of solid state forms having distinct crystal structures and physical properties like melting point, x-ray diffraction pattern, infrared absorption fingerprint, and solid state NMR spectrum. One solid state form may give rise to thermal behavior different from that of another solid state form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

The discovery of new polymorphic forms of Erlotinib provides a new opportunity to improve the performance of the synthesis of the active pharmaceutical ingredient (API) by producing solid state forms of Erlotinib having improved characteristics, such as flowability, and solubility. Thus, there is a need in the art for polymorphic forms of Erlotinib.

### SUMMARY OF THE INVENTION

In one embodiment, the invention encompasses amorphous erlotinib.

In another embodiment, the present invention encompasses a process for preparing amorphous erlotinib comprising lyophilizing a solution of Erlotinib in 1,4-dioxane.

In another embodiment, the present invention encompasses a process for preparing an Erlotinib crystalline form characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 3, comprising heating crystalline erlotinib form, characterized by data selected from the group consisting of: an X-ray powder diffraction pattern with peaks at about 7.5, 10.9, 11.3, 14.7, 15.0 and 24.8 degrees two-theta±0.2 degrees two-theta, and a PXRD pattern as depicted in FIG. 1, to a temperature of about 70° C. to about 120° C.

In another embodiment, the present invention encompasses a process for preparing an Erlotinib crystalline form characterized by data selected from the group consisting of: an X-ray powder diffraction pattern with peaks at about 7.5, 10.9, 11.3, 14.7, 15.0 and 24.8 degrees two-theta±0.2 degrees two-theta, and a PXRD pattern as depicted in FIG. 1, comprising reacting an aqueous mixture comprising of Erlotinib salt with a base providing a suspension comprising the said crystalline form.

In another embodiment, the present invention encompasses a process for preparing an Erlotinib crystalline form characterized by data selected from the group consisting of: an X-ray powder diffraction pattern with peaks at about 6.9, 8.9, 13.2, 13.6 and 24.2 degrees two-theta±0.2 degrees two-theta, and a PXRD pattern as depicted in FIG. 5, comprising reacting 4-chloro-6,7-bis(2-methoxy-ethoxy)quinazoline of the following formula

### 4-Chloro-4,7-Bis-(2-methoxyethoxy)quinazoline

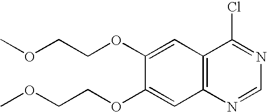

3-ethynylaniline of the following formula

### 3-Ethynylbenzamine

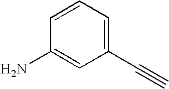

and an alkali hydroxide in isopropanol (“IPA”).

In another embodiment, the present invention encompasses the preparation of erlotinib salt comprising amorphous erlotinib base. Preferably, the erlotinib salt is erlotinib hydrochloride.

In another embodiment, the present invention encompasses a process for preparing an erlotinib salt, comprising preparing any one of the above polymorphs of erlotinib, or a mixture thereof, according to the processes of the present invention and converting it to an Erlotinib salt. Preferably, the erlotinib salt is erlotinib hydrochloride.

### BRIEF DESCRIPTION OF THE DRAWINGS

FIG. 1. illustrates an X-ray powder diffraction pattern of crystalline form G1 of Erlotinib.

FIG. 2. illustrates a DSC curve of the crystalline form G1 of Erlotinib.

FIG. 3. illustrates an X-ray powder diffraction pattern of crystalline form G2 of Erlotinib.

FIG. 4. illustrates a DSC curve of the crystalline form G2 of Erlotinib

FIG. 5. illustrates an X-ray powder diffraction pattern of crystalline form G3 of Erlotinib.

FIG. 6. illustrates a DSC curve of the crystalline form G3 of Erlotinib.

FIG. 7. illustrates an X-ray powder diffraction pattern of amorphous Erlotinib.

### DETAILED DESCRIPTION OF THE INVENTION

As used herein, the term “erlotinib” refers to erlotinib base of the following formula.

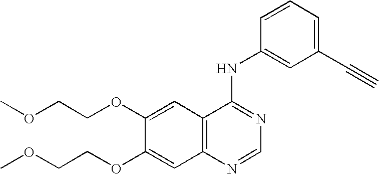

The present invention is directed to amorphous erlotinib, processes for the preparation thereof, and processes for preparing additional forms of erlotinib.

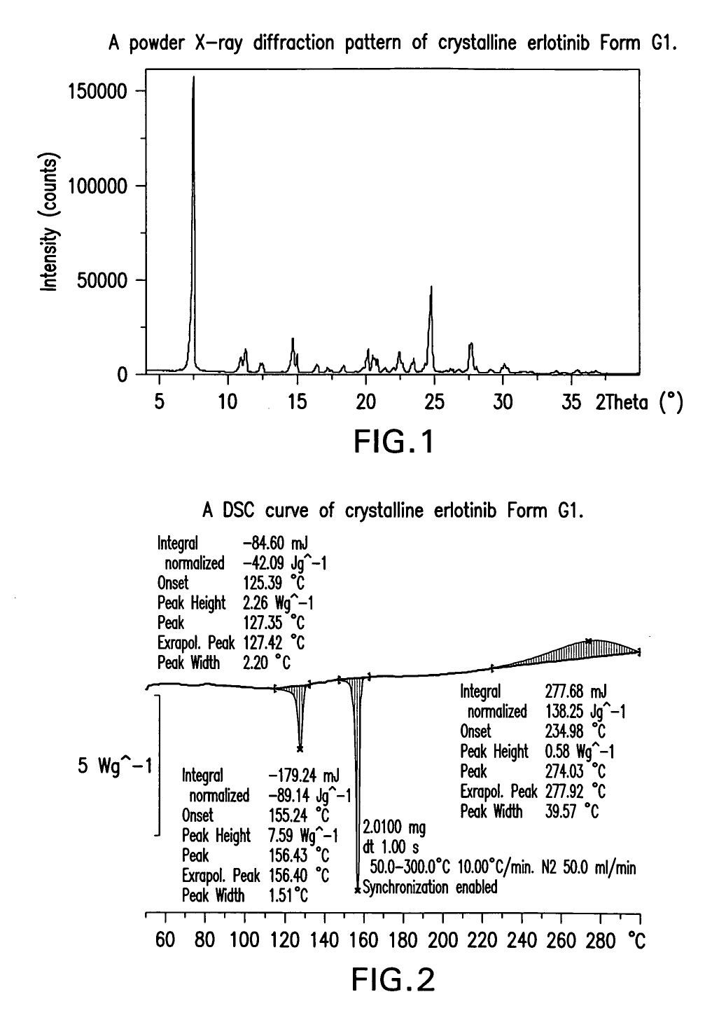

As used herein, the term “crystalline Erlotinib form G1” refers to crystalline Erlotinib characterized by data selected from the group consisting of: an X-ray powder diffraction pattern with peaks at about 7.5, 10.9, 11.3, 14.7, 15.0 and 24.8 degrees two-theta±0.2 degrees two-theta, and a PXRD pattern as depicted in FIG. 1.

The above crystalline form G1 can be further characterized by data selected from the group consisting of: an X-ray powder diffraction pattern having peaks at about 12.4, 20.2, 22.4 and 27.8 degrees two-theta±0.2 degrees two-theta, a DSC thermogram having an endothermic peak at about 127° C. and another peak at 156° C.; and a DSC curve as depicted in FIG. 2.

The said crystalline form G1 is a hydrated form of erlotinib, preferably, a monohydrated form. The water content of the said crystalline form is preferably about 4% to about 5% by weight, more preferably, about 4.4% by weight, as measured by KF or by TGA.

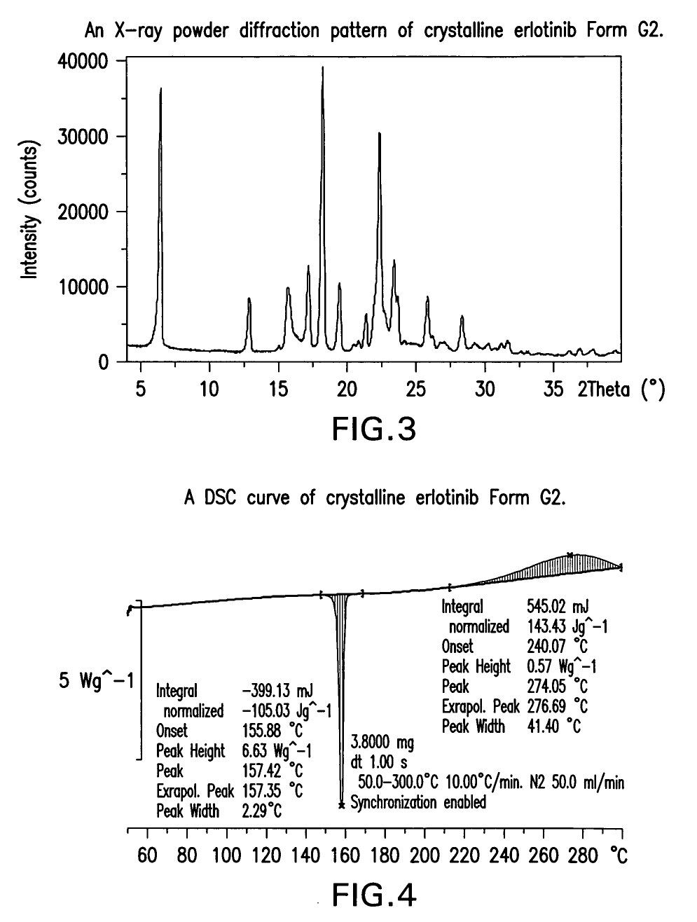

As used herein, the term “crystalline Erlotinib form G2” refers to crystalline Erlotinib characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 3.

The above crystalline form G2 can be further characterized by data selected from the group consisting of: an X-ray powder diffraction pattern having peaks at about 15.7, 19.5, 23.5, 23.7 and 25.9 degrees two-theta±0.2 degrees two-theta, a DSC thermogram having an endothermic peak at about 157° C.; and a DSC curve as depicted in FIG. 4.

The said crystalline form is an anhydrous form of erlotinib. The water content of the said crystalline form is up to about 0.3% by weight, more preferably, about 0.12% by weight, as measured by KF.

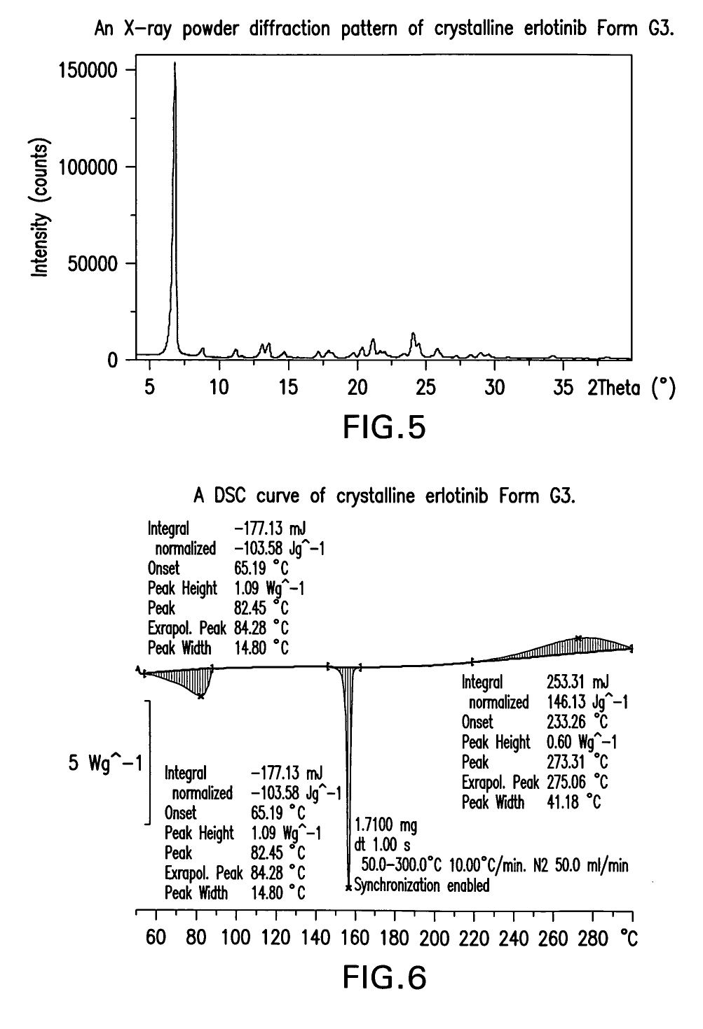

As used herein, the term “crystalline Erlotinib form G3” refers to crystalline Erlotinib characterized by data selected from the group consisting of: an X-ray powder diffraction pattern with peaks at about 6.9, 8.9, 13.2, 13.6 and 24.2 degrees two-theta±0.2 degrees two-theta, and a PXRD pattern as depicted in FIG. 5.

The above crystalline form G3 can be further characterized by data selected from the group consisting of: an X-ray powder diffraction pattern having peaks at about 11.3, 14.7, 17.3, 18.0 and 21.2 degrees two-theta±0.2 degrees two-theta, a DSC thermogram having an endothermic peak at about 82° C. and a second endothermic peak at about 156° C.; and a DSC curve as depicted in FIG. 6.

The said crystalline form is a hydrated form of erlotinib, preferably, a monohydrated form. The water content of the said crystalline form is of about 4% to about 5% by weight, more preferably, of about 4.53% by weight, as measured by KF or by TGA.

In one embodiment, the invention encompasses amorphous erlotinib.

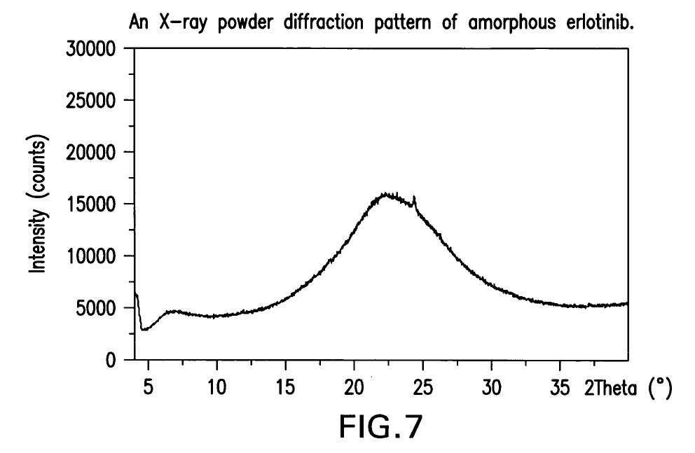

The amorphous erlotinib can be characterized by an X-ray powder diffraction pattern as depicted in FIG. 7. Amorphous erlotinib may be identified by the absence of any significant diffraction peak at the X-ray powder diffraction pattern.

Amorphous Erlotinib can be prepared by a process comprising: lyophilizing a solution of Erlotinib in 1,4-dioxane.

The process comprises providing a solution of erlotinib in 1,4-dioxane and lyophilizing the solution.

Preferably, the solution is provided by a process comprising combining erlotinib and 1,4-dioxane and heating the combination. Preferably, the heating is to a temperature in the range of about 40° C. to about 101° C., more preferably to a temperature of about 80° C.

Preferably, the lyophilization comprises cooling the solution and evaporating the solvent. Preferably the cooling is done gradually. First, cooling to a temperature in the range of about 10° C. to about 40° C., preferably to a temperature of about 25° C. is done, and then cooling to a temperature in the range of about +11.8° C. to about −40° C., preferably to a temperature of about −30° C. is performed.

Typically, the evaporation of the solvents is done at a temperature in the range of about +11.8° C. to about −40° C., preferably to a temperature at about −30° C. Preferably, evaporation of the solvent is done under reduced pressure. Preferably, the reduced pressure is of about 1 mBar.

The above amorphous erlotinib can be used to prepare erlotinib salt, preferably, the HCl salt.

The preparation can be done by reacting amorphous erlotinib and the corresponding acid. When the salt is HCl, the preparation can be done for example, according to the process disclosed in U.S. Pat. No. 5,747,498.

The crystalline Erlotinib form G2 can be prepared by a process comprising heating crystalline erlotinib form G1 to a temperature of about 70° C. to about 120° C.

Preferably, the heating is to a temperature of about 80° C. to about 110° C., more preferably of about 80° C. to about 100° C.

Typically, the starting crystalline Erlotinib is heated for a sufficient time to allow the transformation to the crystalline Erlotinib form G2. Preferably, the heating is done for about 5 to about 20 hours, more preferably, for about 12 hours.

The obtained erlotinib form G2 has less than about 10% by weight, more preferably less than about 5% by weight, and most preferably less than about 1% by weight, of crystalline erlotinib form G1, crystalline erlotinib form G3 or mixture thereof.

Typically, the content of the crystalline erlotinib selected from a group consisting of: crystalline erlotinib having an X-ray powder diffraction pattern with peaks at about 7.5, 11.3, 14.7 and 24.8 degrees two-theta±0.2 degrees two-theta, and of crystalline erlotinib having an X-ray powder diffraction pattern with peaks at about 6.8, 8.8, 11.2 and 13.6 degrees two-theta±0.2 degrees two-theta is measured by PXRD.

The crystalline form G1 of Erlotinib can be prepared by a process comprising reacting an aqueous mixture comprising ERL salt with a base providing a suspension comprising the said crystalline form.

Preferably, the Erlotinib salt is ERL HCl or ERL mesylate, more preferably, ERL HCl.

Preferably, the aqueous mixture of ERL salt is a suspension, which is provided by suspending ERL salt in water.

To this suspension is then added a base. Preferably, the base is an inorganic base, more preferably, either ammonia or an alkali base. Preferably, the alkali base is sodium hydroxide. The base can be neat or in a form of an aqueous solution. Preferably, sodium hydroxide is used in form of an aqueous solution. Preferably, the concentration of the aqueous solution of the alkali hydroxide is of about 10% to about 60%, more preferably, of about 50% by weight.

Typically, ammonia can be used as a gas or as an aqueous solution. Preferably, an aqueous solution of ammonia is used, i.e., NOH. Preferably, the concentration is of about 5% to about 40%, more preferably, of about 25% by weight.

The addition of the base typically transforms the ERL salt to ERL, which precipitates in a form of crystals. Preferably, the addition of the base provides a pH of about 9 to about 12, depending on the base. If an alkali hydroxide is used, the preferred pH is of about 11 to about 12, and if ammonia is used, the preferred pH is of about 9 to about 10.

Preferably, the suspension is maintained for about 0.5 hours to about 50 hours, more preferably for about 2 hours, to form granulates. Preferably, the suspension is maintained under agitation.

Optionally, the obtained erlotinib can be extracted to an organic phase by using an organic solvent such as esters like ethylacetate.

The organic phase is then evaporated providing a residue which is re-slurred in the same solvent providing a precipitate of the said crystalline form G1.

The crystalline Erlotinib can be recovered from the slurry in ethylacetate or from the suspension in water. The recovery can be done by for example by filtering and washing the filtered crystalline form.

The obtained erlotinib form G1 has less than about 10% by weight, more preferably less than about 5% by weight, and most preferably less than about 1% by weight of crystalline erlotinib having a PXRD diffraction pattern with peaks at about 6.5, 12.9, 17.3, 18.3, and 22.4±0.2 degrees two-theta, designated form G2, or crystalline erlotinib having a PXRD diffraction pattern with peaks at about 6.9, 8.9, 13.2, 13.6 and 24.2±0.2 degrees two-theta, designated form G3 of erlotinib, or mixture thereof.

Typically, the content of the crystalline erlotinib selected from a group consisting of: crystalline erlotinib having an X-ray powder diffraction pattern with peaks at about 6.5, 12.9 and 15.7 degrees two-theta±0.2 degrees two-theta, and of crystalline erlotinib having an X-ray powder diffraction pattern with peaks at about 6.8, 8.8, 13.1 and 13.6 degrees two-theta±0.2 degrees two-theta is measured by PXRD.

The above form has well defined crystals and thus, can be easily recovered by filtration. The well defined crystals also contribute to a smaller surface area and thus, to lower absorption of impurities from the mother liquor when precipitated.

The crystalline Erlotinib form G3 can be prepared by a process comprising reacting 4-chloro-6,7-bis(2-methoxy-ethoxy)quinazoline of the following formula

### 4-Chloro-6,7-Bis-(2-methoxyethoxy)quinazoline

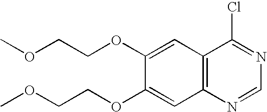

3-ethynylaniline of the following formula

### 3-Ethynylbenzamine

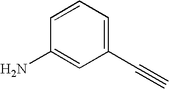

and an alkali hydroxide in isopropanol (“IPA”).

In a preferred embodiment, initially, a suspension of 4-chloro-6,7-bis(2-methoxy-ethoxy)quinazoline and 3-ethynylaniline in EPA is heated. Preferably, the heating is to reflux temperature. Typically, the heating aids in the formation of Erlotinib HCl. Preferably, the heating is done for about 30 minutes.

The heating, typically, provides another suspension, comprising of Erlotinib HCl. This suspension is then combined with IPA and with an alkali hydroxide providing a mixture comprising crystalline erlotinib form G3, which is the free base form.

Preferably, the alkali hydroxide is sodium hydroxide. The alkali hydroxide can be used in a solid or solution form. Preferably, the alkali hydroxide is used in a form of a solution, more preferably, of an aqueous solution. Preferably, the concentration of the aqueous solution of the alkali hydroxide is of about 1N.

The obtained mixture is then maintained for about 30 minutes.

The obtained crystalline Erlotinib can then be recovered from the mixture. The recovery can be done by, for example, filtering the suspension.

The obtained erlotinib form G3 has less than about 10% by weight, more preferably less than about 5% by weight, and most preferably less than about 1% by weight, of crystalline erlotinib form G1, crystalline erlotinib form G2 or mixture thereof.

Typically, the content of the crystalline erlotinib selected from a group consisting of: crystalline erlotinib having an X-ray powder diffraction pattern with peaks at about 7.5, 11.0, 16.5 and 24.8 degrees two-theta±0.2 degrees two-theta, and crystalline erlotinib having an X-ray powder diffraction pattern with peaks at about 6.5, 12.9 and 19.5 degrees two-theta±0.2 degrees two-theta is measured by PXRD.

The above forms as obtained by the processes of the present invention can be used to prepare erlotinib salt, preferably, erlotinib HCl.

The conversion of erlotinib to erlotinib salt can be done by reacting erlotinib and the corresponding acid. When the salt is HCl, the conversion can be done for example, according to the process disclosed in U.S. Pat. No. 5,747,498.

### EXAMPLES

### Instruments

### PXRD

XRPD diffraction was performed on X-Ray powder diffractometer: Philips X'pert Pro powder diffractometer, Cu-tube, scanning parameters: CuKα radiation, λ=1.5419 Å. Continuous scan at a rate of: 0.1° 2 theta/sec. Prior to analysis the samples were gently ground by means of mortar and pestle in order to obtain a fine powder. The ground sample was adjusted into a cavity of the sample holder and the surface of the sample was smoothed by means of a cover glass.

### DSC

DSC measurements were performed on Differential Scanning Calorimeter DSC823e (Mettler Toledo). Al crucibles 40 μl with PIN were used for sample preparation. Usual weight of sample was 1-3 mg.

Program: temperature range 50° C.-300° C., 10° C./min.

### TGA

TGA measurements were performed on instrument TGA/SDTA 851e (Mettler Toledo). Alumina crucibles 70 μl were used for sample preparation. Usual weight of sample was 8-12 mg.

Program: temperature range 50° C.-300° C., 10° C./min.

### Water Content

Water content was determined by Karl Fischer titrator TITRANDO 841, software Tiamo 1.1 (Metrohm). Solution used for determination: Hydranal Composite 2 (Riedel de Haen). Sampling: 100.00 mg, 2 repeats.

### Example 1

### Preparation of Crystalline Erlotinib Form G1

Erlotinib hydrochloride (10.0 g) was suspended in water (60 ml) and 50% NaOH was added stepwise to the suspension under pH-control. The suspension passed through heavy dense stages in the pH range of 5-10. It reached pH between 11-12 after addition of 3 ml NaOH solution. The suspension was transferred into 500 ml bulb and ethylacetate (300 ml) was added. The pH decreased promptly to the value of 5-6 on account of alkaline ethylacetate decomposition. The mixture was heated to the reflux on rotary evaporator (RVO)—the solid phase disappeared and two liquid phases were separated. The water phase was re-extracted again with another portion of ethylacetate (300 ml). The organic layers were combined, evaporated to dryness and crystalline evaporation residue was re-slurred into small amount of ethylacetate (30 ml). The crystalline solids were filtrated off, rinsed with ethylacetate (10 ml) and dried in a small laboratory oven under nitrogen stream (150 l/hr) at 40° C./5 hrs. The yield was 96.3% (9.22 g). The obtained substance is a coarse and creamy powder. This is Erlotinib base monohydrate form G1 according to solid state analyses. (KF 4.39%).

### Example 2

### Preparation of Crystalline Erlotinib Form G1

Erlotinib hydrochloride (20.0 g) was suspended in water (800 ml) and 25% ammonia solution (11 ml) was added slowly to the suspension under pH-control. It reached pH 9.4 at the time when ammonia addition was completed. The suspension was agitated for an additional 2 hrs. Then the crystalline solids were filtrated off, rinsed with water (400 ml) and dried in a small laboratory oven under nitrogen stream (150 l/hr) at 40° C./4 hrs. The molar yield was 94.3% (18.94 g, a creamy powder). The Erlotinib base obtained by the described procedure is monohydrate form G1.

### Example 3

### Preparation of Crystalline Erlotinib Form G2

Erlotinib base monohydrate was treated by heating at 100° C. in a small laboratory oven under nitrogen stream (1501/hr). The thermal exposition lasted 12 hrs. It was obtained as Erlotinib free base form G2 (8.89 g) (KF 0.12%).

### Example 4

### Preparation of Crystalline Erlotinib Form G3

A suspension of 4-chloro-6,7-bis(2-methoxy-ethoxy)quinazoline (5.00 g) and 3-ethynylaniline (2.06 g) in IPA (100 mL) was stirred under reflux for 30 min. The resulting thick suspension was diluted with IPA (100 mL) and added to 1N NaOH (1000 mL). The mixture was stirred for 30 min. The precipitate was collected and dried in vacuum at 40° C. to furnish Erlotinib free base form G3 as a colorless solid (5.68 g; KF 4.53%; 85% yield)

### Example 5

### Procedure for the Preparation of Amorphous Erlotinib Base

Erlotinib base (127 mg) was dissolved in 1,4-dioxane (6 ml) at 80° C. The solution was allowed to cool to 25° C. and put in the refrigerator at −30° C. where the solution was frozen. The frozen solution was transferred to the lyophylisator and a vacuum of 1 mBar was applied, which provided the freeze drying of 1,4-diolane affording amorphous erlotinib base.

### Example 6

### The Conversion of Erlotinib Base to Erlotinib Salt According to U.S. Pat. No. 5,747,498

Erlotinib base was dissolved in minimum volume of CHCl₃, diluted with several volumes of ether, and titrated with 1M HCl in ether to precipitate the title product as its hydrochloride salt.

What is claimed is:

1. Amorphous erlotinib.

2. The amorphous erlotinib of claim 1, characterized by an X-ray powder diffraction pattern as depicted in FIG. 7.

3. A method for preparing amorphous erlotinib, comprising lyophilizing a solution of erlotinib in 1,4-dioxane.

4. The method of claim 3, wherein said solution is provided by a process comprising combining erlotinib and 1,4-dioxane and heating the combination.

5. The method of claim 4, wherein said heating is carried out to a temperature of about 40° C. to about 110° C.

6. The method of claim 3, wherein said lyophilizing comprises cooling the solution and evaporating the solvent.

7. The method of claim 6, wherein said cooling is carried out by a method comprising first cooling to a temperature of about 40° C. to about 10° C., then cooling to a temperature of about +11.8° C. to about −40° C.

8. The method of claim 7, wherein said lyophilizing comprises evaporating said solvent at about +11.8° C. to about −40° C.

9. The method of claim 8, wherein said evaporating is carried out under a reduced pressure of about 1 mBar.

10. A method for preparing an erlotinib salt comprising providing an amorphous erlotinib, and converting said amorphous erlotinib to an erlotinib salt.

11. A method for preparing a crystalline form of erlotinib (ERL) characterized by an X-ray powder diffraction pattern with peaks at about 6.5, 12.9, 17.3, 18.3 and 22.4 degrees two-theta±0.2 degrees two-theta, comprising heating an crystalline erlotinib form characterized by an X-ray powder diffraction pattern with peaks at about 7.5, 10.9, 11.3, 14.7, 15.0 and 24.8 degrees two-theta±0.2 degrees two-theta to a temperature of about 70° C. to about 120° C.

12. The method of claim 11, comprising heating said crystalline erlotinib form characterized by an X-ray powder diffraction pattern with peaks at about 7.5, 10.9, 11.3, 14.7, 15.0 and 24.8 degrees two-theta±0.2 degrees two-theta to a temperature of about 80° C. to about 110° C.

13. The method of claim 11, wherein said heating said crystalline erlotinib form is carried out for a sufficient period of time to allow transformation of said crystalline erlotinib form characterized by an X-ray powder diffraction pattern with peaks at about 7.5, 10.9, 11.3, 14.7, 15.0 and 24.8 degrees two-theta±0.2 degrees two-theta to the crystalline erlotinib form characterized by an X-ray powder diffraction pattern with peaks at about 6.5, 12.9, 17.3, 18.3 and 22.4 degrees two-theta±0.2 degrees two-theta.

14. The method of claim 13, wherein said heating is carried out for about 5 to about 20 hours.

15. A method for preparing a crystalline form of erlotinib (ERL) characterized by an X-ray powder diffraction pattern with peaks at about 7.5, 10.9, 11.3, 14.7, 15.0 and 24.8 degrees two-theta±0.2 degrees two-theta, comprising reacting an aqueous mixture comprising an erlotinib salt with a base to provide a suspension comprising said crystalline form of erlotinib.

16. The method of claim 15, wherein said erlotinib salt is erlotinib HCl or erlotinib mesylate.

17. The method of claim 16, wherein said erlotinib salt is erlotinib HCl.

18. The method of claim 15, wherein said mixture is prepared by suspending said erlotinib salt in water.

19. The method of claim 15, wherein said base is an inorganic base.

20. The method of claim 19, wherein said base is an alkali base.

21. The method of claim 20, wherein said alkali base is sodium hydroxide.

22. The method of claim 20, wherein said alkali base provides a pH of about 11 to about 12.

23. The method of claim 19, wherein said base is ammonia.

24. The method of claim 23, wherein said ammonia is a gas or an aqueous ammonia solution.

25. The method of claim 24, wherein said ammonia is an aqueous ammonia solution.

26. The method of claim 24, wherein said ammonia provides a pH of about 9 to about 10.

27. The method of claim 15, further comprising
a) extracting erlotinib to an organic phase using an organic solvent;
b) evaporating said organic solvent to obtain a residue; and
c) slurrying said residue in said organic solvent; and
d) precipitating said crystalline erlotinib form.

28. The method of claim 27, wherein said organic solvent is an ester.

29. The method of claim 28, wherein said ester is ethyl acetate.

30. A method for preparing a crystalline form of erlotinib (ERL) characterized by an X-ray powder diffraction pattern with peaks at about 6.9, 8.9, 13.2, 13.6 and 24.2 degrees two-theta±0.2 degrees two-theta, comprising reacting 4-chloro-6,7-bis(2-methoxy-ethoxy)quinazoline of the following formula
4-Chloro-6,7-Bis-(2-methoxyethoxy)quinazoline 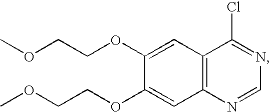
3-ethynylaniline of the following formula
3-Ethynylbenzamine 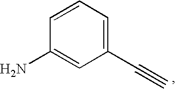
and an alkali hydroxide in isopropanol (IPA).

31. The method of claim 30, wherein said reaction comprises
a) heating a suspension of 4-chloro-6,7-bis(2-methoxy-ethoxy)quinazoline and 3-ethynylaniline in IPA to produce a suspension comprising ERL HCl; and b) combining said suspension with said alkali hydroxide.

32. The method of claim 31, wherein said heating is carried out at about reflux temperature.

33. The method of claim 30, wherein said alkali hydroxide is sodium hydroxide.

34. The method of claim 30, wherein said alkali hydroxide is in a solid or solution form.

35. The method of claim 34, wherein said alkali hydroxide is an alkali hydroxide solution.

36. The method of claim 35, wherein said alkali hydroxide solution is an aqueous solution.

37. The method of claim 30, further comprising recovering crystalline erlotinib from said mixture.

38. A method for preparing an erlotinib salt, comprising
a) preparing erlotinib forms according to the method of any of claims 3, 11, 15, or 30; and
b) converting said crystalline erlotinib form prepared in step a) to said erlotinib salt.

39. The method of claim 38, wherein the salt is HCl.
